(19) United States
(12) Patent Application Publication
(10) Pub. No.: US 2002/0077517 A1
(43) Pub. Date: Jun. 20, 2002

(54) Method and system for removing ethylbenzene and non-aromatics from a mixed xylene stream and optimizing para-xylene production and separation

(76) Inventors: Goerge R. Winter, Fond Du Lac, WI (US); Zvi Merchav, Haifa (IL)

Correspondence Address: Kajane McManus, P.O. Box 344, Wonder Lake, IL, 60097, US

(21) Appl. No.: 09/902,404

(22) Filed: Jul. 10, 2001

Related U.S. Application Data
(63) Continuation-in-part of application No. 08/999,993, filed on May 6, 1996, now abandoned.
Application No. 08/999,993 is a continuation-in-part of application No. 08/437,986, filed on May 10, 1995.

(51) Int. Cl. C07C 5/25; C07C 7/00

(52) U.S. Cl. 585/479; 585/478; 585/481; 585/483; 585/805

(57) ABSTRACT

The system and method for optimizing para-xylene production from a feed stream containing para-xylene, ortho-xylene, meta-xylene, ethylbenzene and non-aromatics propose directing the feed stream into an isomerization unit of the system rather than to a separator of the system to avoid causing a bottleneck at the separator and to initially remove ethylbenzene and non-aromatics from the feed stream. This system and method may further provide a reactor in the feed stream used in a pretreatment step, the reactor containing an isomerization catalyst in an amount sufficient to convert substantially all the ethylbenzene in the feed stream to benzene, which is immediately removed and to convert and remove non-aromatics therefrom to optimize equilibrium isomer concentration in the isomerization unit and substantially eliminate coking therein.

### CROSS REFERENCE TO RELATED APPLICATION

This application is a continuation-in-part of U.S. patent application Ser. No. 08/999,993 filed May 6, 1996 which is a continuation-in-part of application Ser. No. 08/437,986 filed May 10, 1995 and entitled Method and System for Removing Ethylbenzene from a Para-xylene Feed Stream.

### BACKGROUND OF THE INVENTION

1. Field of The Invention

The present invention provides a method and system for removing ethylbenzene and non-aromatics from a mixed xylene stream in a reactor positioned to accept raw feed and empty into an isomerization unit for optimizing para-xylene production and separation. More specifically the method and system propose directing flow of a para-xylene feed stream into an isomerization unit of the system rather than to a para-xylene separator of the system to avoid a bottleneck condition from being created at the separator. If desired, a pretreatment step may be included which comprises processing the feed stream through a reactor comprising a small version of an isomerization unit within which an isomerization catalyst is provided, the catalyst causing a high level of conversion of ethylbenzene to benzene and conversion and removal of most, if not all, non-aromatics while minimizing side reactions, such as cracking of xylenes. The benzene, together with the non-aromatics, is thus removed from the feed stream prior to cyclic isomerization and para-xylene separation, respectively.

Although the system and method are proposed for use in para-xylene production, they are equally well suited to use in production of ortho-xylene and meta-xylene.

2. Description of the Prior Art

Para-xylene is a precursor in the manufacture of polyesters, which are used in creating clothing and other synthetic materials. Most para-xylene is produced in oil refineries downstream of catalytic reformers which manufacture gasoline.

Gasoline contains a mixture of hydrocarbons including C8 aromatics. The C8 aromatics include four chemical compounds: para-xylene, meta-xylene, ortho-xylene and ethylbenzene. The para-xylene is isolated and extracted from the other C8 aromatics by one of two physical separation processes, crystallization or mol sieve technology. Once the para-xylene extract has been removed from the mixture of C8 aromatics, the remaining mixture of meta-xylene, ortho-xylene, ethylbenzene and non-aromatics, commonly referred to as raffinate, is sent to an isomerization unit where further para-xylene is created by returning the xylene mixture to an equilibrium concentration, with the new ratio of xylene isomers, commonly referred to as effluent, being returned to the separation unit for reprocessing.

The concentration of para-xylene in the total feed entering the separation unit determines the efficiency of the operation. For example, in a crystallizer, the mother liquor contains about 10% para-xylene. Therefore, if the total feed stream contains 19%, about 9% will be extracted and 10% will not be extracted. However, if the total feed stream contains 21% para-xylene, the production rate increases substantially due to an 11% extraction. Thus, increasing the para-xylene in the feed stream from 19% to 21% will increase the plant capacity by approximately 11/9, or 20%. An optimum goal is to provide an enriched 24% para-xylene effluent to a high efficiency separator.

In modern units, the ethylbenzene is dealkylated to benzene in the isomerization unit. This reaction proceeds at 50 to 60% conversion per pass. Thus, the feed stream provided to the separation unit always contains a substantial amount of ethylbenzene as well as non-aromatics. The ethylbenzene, together with the non-aromatics, builds up in the combined mother liquor/raffinate stream requiring processing equipment to be larger than necessary to process the para-xylene, ortho-xylene and meta-xylene. Since the feed stream to the separation unit contains approximately 20% para-xylene, the mother liquor/raffinate stream will contain the unextracted para-xylene plus the remainder of the chemicals which are not para-xylene. Thus, the mother liquor/raffinate stream becomes quite large, requiring large processing equipment. For a crystallizer, the mother liquor recycle is six times para-xylene flow rate. In a mol sieve extraction unit, the raffinate is about four times the para-xylene flow rate. If ethylbenzene and non-aromatics were removed from the feed stream initially, the remaining stream would have substantially higher concentrations of para-xylene, meta-xylene and ortho-xylene therein. Also, when the feed stream is directed into a less efficient para-xylene separator, together with the recycled feed or effluent returning to the separator from the isomerization unit, a bottleneck occurs within the separator, slowing the separation process, thereby producing a lesser amount of para-xylene over time.

Further, as alluded to above, there is not only ethylbenzene to be dealt with as a “contaminant” but non-aromatics (non-aromatic hydrocarbons) as well. Because of the presence of such non-aromatics, conditions in upstream catalytic reformers must be severe.

By removing a high percentage of the non-aromatics from the stream, through use of the proposed pretreatment reactor, severity of conditions in the upstream catalytic reformers is significantly reduced. Such benefit typically provides higher gasoline yield, higher overall system capacity and less coking, all being desirable qualities in the process.

Still further, in the cyclic loop of isomerization/separation, another benefit is obtained. Since coking in the isomerization unit is a function of the amount of ethylbenzene present, with removal of ethylbenzene and non-aromatics by the pretreatment to a substantially zero level, energy consumption and capital costs are dramatically decreased.

### SUMMARY OF THE INVENTION

Accordingly, it is a primary object of the method and system of the present invention to remove substantially all ethylbenzene and non-aromatics from the system as an initial step of the method and further to direct the feed stream to the isomerization unit rather than to the separator of the system.

Further, if desired to remove ethylbenzene and non-aromatics from the feed stream in a pretreatment step to create a maximized point of xylene isomer equilibrium in the isomerization unit which revolves only around three xylenes by decreasing coking in the isomerization unit, optimizing the system, a pretreatment reactor, which is substantially a small isomerization unit, may be provided in the feed stream flow path, upstream of the isomerization unit. Finally, by manipulating the feed location and operating variables, more beneficial reactions can be accomplished in an isomerization reactor processing both fresh feed and mother liquor/raffinate stream, increasing yield.

### BRIEF DESCRIPTION OF THE DRAWINGS

FIG. 1 is a block diagram of a prior art method and system for obtaining para-xylene product.

FIG. 2 is a block diagram of a recently proposed improved method and system for obtaining para-xylene product.

FIG. 3 is a block diagram of the method and system of the present invention incorporating a pretreatment reactor.

FIG. 4 is a block diagram of the method and system of the present invention without a pretreatment reactor.

### DESCRIPTION OF THE PREFERRED EMBODIMENT

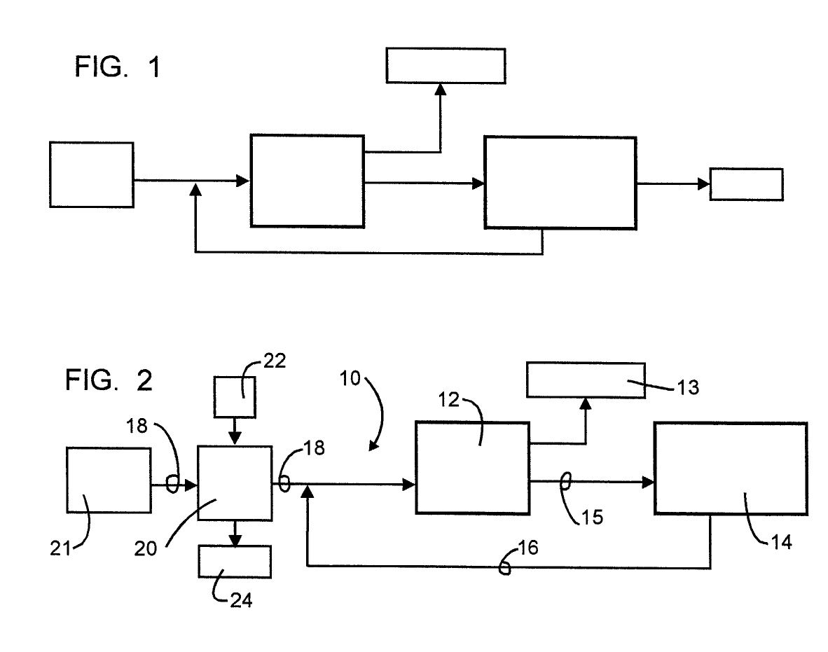

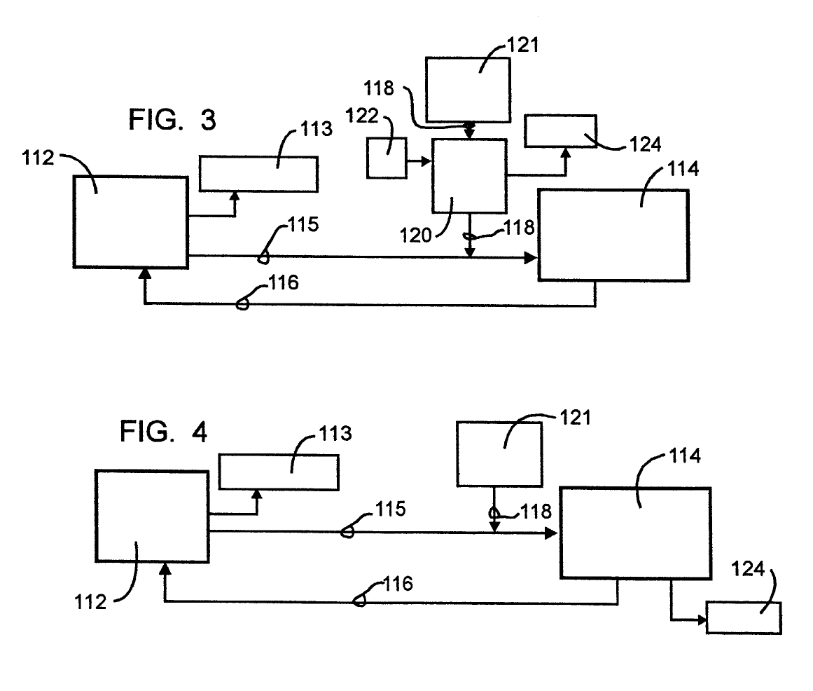

Turning now to FIG. 1 there is illustrated therein a prior art method and system for the production of para-xylene product. As shown, a feed stream comprising a mixture of eight-carbon (C8) aromatics including para-xylene, meta-xylene, ortho-xylene, ethylbenzene and non-aromatics is fed to a para-xylene separator. From the separator, existing para-xylene is shunted into a collection unit and a raffinate containing non-aromatics, ethylbenzene, ortho-xylene, and meta-xylene is passed on to an isomerization unit. In this isomerization unit, the ethylbenzene, which causes coking in the unit, is dealkylated to benzene under high severity conditions with the reaction proceeding at only 50 to 60% conversion per pass. The converted benzene, together with some of the non-aromatics converted to light petroleum gases, is removed from the stream and an effluent comprising the remainder of the recycle feed stream, now containing somewhere in the range of 40 to 50% of the previous amount of ethylbenzene and substantial amounts of remaining non-aromatics therein, together with equilibrium amounts of ortho-xylene, meta-xylene and para-xylene formed in the isomerization unit, is sent back to the feed line via which the feed stream is being fed into the para-xylene separator.

Cyclic separation and isomerization continues until the feed is completely consumed.

It will be understood that the ethylbenzene, together with the non-aromatics, builds up in the recycle feed stream since the recycle stream to the separation unit typically contains 20% para-xylene with the feed stream containing unextracted para-xylene plus the 80% comprising the other C8 aromatics defined above. Thus, the recycle stream is quite large, requiring processing equipment for this process to be extremely large to accommodate the “contaminated” flow therethrough that is being created.

Thus, if one were capable of removing the ethylbenzene and non-aromatics from the feed in a pretreatment step, rather than removing it in the isomerization unit under high severity conditions, approximately 15% of the feed stream bulk could be eliminated at the beginning of the process, a greater concentration of para-xylene would be obtained, and the isomerization unit as well as the separation unit would both work more efficiently under less severe conditions. Not only would this increase the efficiency, requiring significantly decreased energy for processing, but further the size of the units could be decreased substantially while maintaining increased processing capacity. Still further, operating conditions in the isomerization unit would be less severe, significantly increasing yield and extending catalyst life.

One such system 10 is shown in FIG. 2, wherein substantially all of the ethylbenzene is converted to benzene and substantially all of the non-aromatics are converted to light petroleum gases in a pretreatment, or primary step, the benzene being distilled, and removed together with the light petroleum gases, with only para-xylene, ortho-xylene and meta-xylene being fed into the separator of the processing complex. For purposes of brevity, application to a para-xylene production complex is set forth as a primary embodiment and a broader scope application would be feasible without undue experimentation. As shown, the system 10 includes a para-xylene separator 12 which functions to extract para-xylene product 13 for collection and an isomerization unit 14 which functions in a more efficient manner due to the provision of a pretreatment reactor 20 in feed line 18, the function of which will be further defined hereinafter.

In this respect, inasmuch as there will be almost no ethylbenzene or non-aromatics being fed into the isomerization unit 14, none of the C8 aromatics being fed thereinto need to be eliminated from the feed stream. Thus, the isomerization unit 14 here strictly functions to produce para-xylene from ortho-xylene and meta-xylene in the raffinate 15 fed thereto with severity of conditions therein being substantially lessened. Obviously, inasmuch as several passes through the isomerization unit 14 will now be, and always have been, required, there typically will be an equilibrium concentration of meta-xylene, ortho-xylene and para-xylene in an effluent line 16 feeding into the feed line 18 to the para-xylene separator 12, downstream of the reactor 20. However, due to increased efficiency because of less severity in conditions and removal of ethylbenzene and non-aromatics from the effluent in line 16, the amount of meta-xylene and ortho-xylene will be significantly reduced, with a greater amount of para-xylene product being available in effluent line 16, since substantially no ethylbenzene or non-aromatics will exist in the effluent line 16.

The ethylbenzene, together with the non-aromatics, is proposed to be removed from the feed stream going into the para-xylene separator 12 by the provision of a pretreatment unit or reactor 20 within the feed line 18 for the fresh feed 21. This pre-treatment unit 20 comprises a small version of an isomerization unit and includes therein a large amount of an isomerization catalyst 22, one form of which is sold under the mark I-100 by UOP, Inc. of Des Plaines, Ill. Other companies that sell similar catalysts are Criterion Co. of Houston, Tex., Englehardt Co., of New York, N.Y., IFP (Institute de Francaise Petroleum), Paris, France, and Toray (Toyo Rayon Company) of Tokyo, Japan. It is believed that such catalyst 22 is created of platinum and chloride supported on alumina. Further, a catalyst which would be based on a molecular sieve base would also be functional, such catalyst being available through Mobil Oil Corporation of Princeton, N.J.

It has been found through empirical testing using the I-100 catalyst that an approximately 90% conversion of ethylbenzene to benzene 24 may be achieved in the pretreatment reactor 20 at a liquid hourly space velocity of 1 to 4, as is known, with the benzene 24 being immediately purged from the system 10. Also, because severity of conditions may be held at a high level in the pretreatment reactor 20 without adverse effects, substantially all of the non-aromatics are converted to light petroleum gases and removed at this point as well. Because the feed stream 18 rate is much lower than the recycle stream or effluent 16 rate, the catalyst 22 volume may be large in comparison to the hydrocarbon rate, such large volume of catalyst permitting a high rate of ethylbenzene conversion and removal thereof, together with conversion and removal of the non-aromatics, while avoiding side reactions such as cracking of xylenes.

Based on calculations founded on the empirical testing performed, the following advantages, based on separation technique, are expected:

1. Mol Sieve Technology

For a para-xylene unit using mol sieve technology making 20 MT/hr of para-xylene product, the total flow rate to the para-xylene separation unit will be 86.3 MT/hr, and the feed rate to the isomerization unit will be about 60 MT/hr.

Using the system 10 and method defined above and holding constant the feed rate to the para-xylene separation unit, para-xylene production will increase to 27.2 MT/hr, an increase of 36%. At the current market price of $2,000/MT, the incremental increase in production is worth over $115 million per year:

(7.2 MT/hr) (8,000 hrs/yr) ($2,000/MT)=$115,200,000/yr.

Because the raffinate 15 fed to the isomerization unit 14 will decrease to 55.3 MT/hr, the energy cost will decrease by almost 8%:

(55.3 MT/hr)/(59.8 MT/hr)=92%

At an energy cost of roughly $100/MT of para-xylene presently existing, this would be worth over $1,000,000 per year:

($100/MT) (20 MT/hr) (8,000 hrs/yr) (8%)=$1,280,000/yr

2. Crystallization Technology

If the para-xylene separation uses crystallization technology, the circulation numbers change, but the results are just as dramatic. Holding the circulation rate at about 60 MT/hr, the production of para-xylene will be about one-half of that produced by the mol sieve unit, or 10 MT/hr.

By use of the system 10 and method defined above, the production of para-xylene will increase to 13.6 MT/hr, half of the above numbers. Likewise, the benefits will be about half of the dollars calculated above.

It will also be understood that some feed stocks for para-xylene production are prepared from low pressure reformers, or high pressure reformers followed by extraction. In low pressure reformers, non-aromatics in the xylene volatility range are reacted to either aromatics or to light non-aromatics. In reformers operating above 100 psig, the non-aromatics do not react to near extinction. As a result, if the system 10 and method are not used, the non-aromatics must be separated from the aromatics by extraction, an expensive process.

A further strong advantage of system 10 and method is that hydrocracking of the non-aromatics to light compounds occurs so that they can easily be removed from the xylenes.

Such system 10 has been found through empirical testing to be very functional in applications where the separator 12 parameters can accommodate combined feed 18 and recycle 16 flow.

However, if separator 12 parameters cannot accommodate the combined flow, a bottleneck condition has been found to occur at the separator 12, producing a less than optimum para-xylene product 13 output.

In such an instance, the system 100 and method of present invention are proposed for use.

The system 100 includes a separator 112 and an isomerization unit 114. Here, however, to avoid a bottleneck condition from forming at the separator 112, the flow path 118 for the feed 121 has been modified to lead into the isomerization unit 114. To maximize isomerization, a pretreatment reactor 120 may be incorporated in the feed flow path 118, to remove substantially all ethylbenzene and non-aromatics from the feed 121, as described hereinabove, again optimizing para-xylene production in the isomerization unit 114. Now, the isomerization unit 114, functioning under less severe conditions, outputs an enriched effluent 116 having an optimized concentration of mixed xylenes.

Now only the enriched mixed xylene effluent 116 is fed to the separator 112, and an optimized amount of xylene product 113 is extracted.

The pretreatment reactor 120 has heretofore been defined as a small version of an isomerization unit functioning under typical high severity operating conditions for such unit and including therein a large amount of an isomerization catalyst. Operating parameters, which are unit specific, may also be easily recalculated by those skilled in the art, if necessary, to optimize ethylbenzene and non-aromatics conversion and removal.

Such pretreatment reactor 120 is proposed for use with an isomerization unit 114 which is small and/or inefficient.

However, where an extremely large and efficient isomerization unit 114 is provided, if desired, pretreatment of the raw feed 121 entering directly into the isomerization unit 114 may be eliminated to save cost, as shown in FIG. 4. Ethylbenzene conversion and extraction, together with conversion and removal of non-aromatics, is still being initially performed, now within the efficient isomerization unit 114 by setting operating parameters thereof to produce an environment therein which mimics that of the reactor 120, with converted benzene 124 and non-aromatics being immediately eliminated from the system 100 through directing of raw feed 121 directly into the isomerization unit 114 rather that into the separator 112. Again, only an enriched mixed xylene effluent 116 is provided to the separator 112.

It will be understood that only a portion of ethylbenzene present has previously been converted to benzene and only a portion of the non-aromatics, have been removed in the isomerization unit 14, shown in FIG. 1, as defined hereinabove. Inasmuch as it is desirable to produce maximized initial dealkylation, the known dealkylation control variables of liquid hourly space velocity, temperature, hydrogen partial pressure and/or catalyst amount for the specific efficient isomerization unit 114 being utilized also may be calculated and set to produce substantially complete ethylbenzene and non-aromatics conversion and removal on the first pass of the feed 121 through the isomerization unit 114.

As defined above, the method and system of the present invention provide a number of advantages, some of which have been described above and others of which are inherent in the invention. Also modifications may be proposed without departing from the teachings herein. For example, if the desired product were ortho-xylene, the separator used would be in the form of a distillation tower, which is considered within the scope of the invention. Further, if a functionally equivalent catalyst is used in place of an isomerization catalyst, this would also be construed as within the scope of the invention. Accordingly, the scope of the invention is only to be limited as necessitated by the accompanying claims.

We claim:

1. A system for use in producing at least one chosen xylene isomer product from a raw feed stream including para-xylene, ortho-xylene, meta-xylene, ethylbenzene and non-aromatics, the system including:
an isomerization unit into which the raw feed stream is directly fed, the isomerization unit having a catalyst therein which converts the feed stream into a substantially non-aromatics free and ethylbenzene-free effluent having optimized equilibrium concentrations of para-xylene, ortho-xylene and meta-xylene; and
at least one xylene isomer separator into which the effluent is fed for extraction of the chosen xylene isomer to produce a raffinate of remaining xylene isomers to be recycled.

2. The system of claim 1 wherein the separator is a molecular sieve.

3. The system of claim 1 wherein the separator is a crystallizer.

4. The system of claim 1 further including a pretreatment reactor in the raw feed stream, the reactor having an isomerization catalyst therein for converting the ethylbenzene to benzene which is immediately eliminated and wherein non-aromatics are converted to light petroleum gases and immediately eliminated as well.

5. The system of claim 4 wherein the isomerization catalyst in the reactor is a catalyst based on a molecular sieve base.

6. The system of claim 4 wherein the isomerization catalyst in the reactor is a catalyst sold under the trademark I-100.

7. The system of claim 1 wherein the isomerization catalyst in the isomerization unit is a catalyst sold under the trademark I-100.

8. A method for producing at least one chosen xylene isomer product from a raw feed stream including ortho-xylene, meta-xylene, para-xylene, ethylbenzene, and non-aromatics, the method comprising the steps of:
passing the feed stream directly to and through an isomerization unit having an isomerization catalyst therein and having unit specific operating conditions of temperature, liquid hourly space velocity, hydrogen partial pressure and/or catalyst amount calculated and set to maximize ethylbenzene and non-aromatic conversion;
converting ethylbenzene to benzene and non-aromatics to light petroleum gases and removing the converted benzene and light petroleum gases from the stream while creating a predefined equilibrium effluent of xylene isomers in the isomerization unit;
passing the effluent into an isomer specific separator;
extracting the desired isomer product; and
passing a raffinate of remaining xylene isomers back to the isomerization unit.

9. The method of claim 8 wherein the effluent is cyclically reprocessed.

10. The method of claim 8 wherein the separator is provided in the form of a crystallizer.

11. The method of claim 8 wherein the separator is provided in the form of a molecular sieve.

12. A method for producing high purity mixed xylenes from an unextracted mixture rich in eight carbon aromatics, the method comprising the steps of:
starting with a feed stream including para-xylene, meta-xylene, ortho-xylene, ethylbenzene and non-aromatic compounds;
feeding the feed stream into a pretreatment reactor having a chosen isomerization catalyst therein in an amount sufficient to cause a chosen level conversion of ethylbenzene to benzene;
immediately removing the converted benzene from the stream within the pretreatment reactor;
concurrently causing a high level conversion of non-aromatic compounds to lighter hydrocarbons within the reactor; and
removing the lighter hydrocarbons from the stream within the pretreatment reactor.

13. A method for producing at least one chosen xylene isomer product from a feed stream including ortho-xylene, meta-xylene, para-xylene, and ethylbenzene, the method comprising the steps of:
starting with a feed stream including para-xylene, meta-xylene, ortho-xylene, and ethylbenzene;
passing the feed stream through a pretreatment reactor having a chosen isomerization catalyst therein in an amount sufficient to cause a high level conversion of ethylbenzene to benzene and immediately removing the converted benzene from the stream, creating an enriched mixed xylene feed stream;
passing the enriched mixed xylene feed stream to and directly through an isomerization unit also having the chosen isomerization catalyst therein to produce an effluent having an optimized predefined equilibrium concentration of xylene isomers therein;
passing the effluent into an isomer specific separator for processing;
extracting the desired isomer from the effluent; and
passing a raffinate of remaining xylenes back to the isomerization unit.

14. The method of claim 13 wherein the effluent is cyclically reprocessed.

15. The method of claim 13 wherein the separator is provided in the form of a crystallizer.

16. The method of claim 13 wherein the separator is provided in the form of a molecular sieve.
